(19) United States
(12) Patent Application Publication
(10) Pub. No.: US 2024/0424148 A1
(43) Pub. Date: Dec. 26, 2024

(54) IN-VIVO PROXIMITY-BASED LABELING SYSTEMS AND APPLICATIONS THEREOF

(71) Applicant: THE TRUSTEES OF PRINCETON UNIVERSITY, Princeton, NJ (US)

(72) Inventors: Jacob B. Geri, Plainsboro, NJ (US); Benito F. Buksh, Princeton, NJ (US); David W.C. MacMillan, Princeton, NJ (US)

(21) Appl. No.: 18/698,529

(22) PCT Filed: Oct. 4, 2022
(86) PCT No.: PCT/US2022/045654

§ 371 Date: Apr. 4, 2024

Related U.S. Application Data
(60) Provisional application No. 63/252,244, filed on Oct. 5, 2021.

(51) Int. Cl. A61K 49/00; G01N 33/80

(52) U.S. Cl. CPC A61K 49/0036; A61K 49/0052; G01N 33/80; G01N 2458/15

(57) ABSTRACT

In one aspect, compositions and methods are described herein for providing a microenvironment mapping platform operable to selectively identify various features, including in vivo protein-protein interactions on cellular membranes. In some embodiments, a composition comprises a tetrapyrrole photocatalyst, and a protein labeling agent, wherein the tetrapyrrole photocatalyst has electronic structure to activate the protein labeling agent to a reactive intermediate via energy transfer. The reactive intermediate reacts or crosslinks with a protein or other biomolecule within the diffusion radius of the reactive intermediate.

### RELATED APPLICATION DATA

The present application claims priority pursuant to Article 8 of the Patent Cooperation Treaty to U.S. Provisional Patent Application Ser. No. 63/252,244 filed Oct. 5, 2021, which is incorporated herein by reference in its entirety.

### FIELD

The present invention relates to proximity-based labeling systems and, in particular, to compositions and methods permitting high resolution labeling of in-vivo biological environments.

### BACKGROUND

Protein proximity labeling has emerged as a powerful approach for profiling protein inter-action networks. The ability to label associated or bystander proteins through proximity labeling can have important implications on further understanding the cellular environment and biological role of a protein or biomolecular species of interest. Current proximity labeling methods all involve the use of enzyme-based generation of reactive intermediates that label neighboring proteins on a few select amino acid residues through diffusion or physical contact. Despite the transformative impact of this technology, the inherent stability of these reactive intermediates such as phenoxy radicals (t_{1/2}>100 μs) through peroxidase activation or biotin-AMP (t_{1/2}>60 s) through biotin ligases can promote diffusion far from their point of origin. As a result, these enzyme-generated reactive intermediates pose a challenge to profiling within tight micro-environments. Furthermore, the large enzyme size, the dependency on certain amino acids for labeling, and the inability to temporally control these labeling systems present additional challenges for profiling within confined spatial regions. Given these limitations, new approaches for proximity-based labeling are needed.

### SUMMARY

In one aspect, compositions and methods are described herein for providing a microenvironment mapping platform operable to selectively identify various features, including in-vivo protein-protein interactions on cellular membranes. In some embodiments, a composition comprises a tetrapyrrole photocatalyst, and a protein labeling agent, wherein the tetrapyrrole photocatalyst has electronic structure to activate the protein labeling agent to a reactive intermediate via energy transfer. The reactive intermediate reacts or crosslinks with a protein or other biomolecule within the diffusion radius of the reactive intermediate. If a protein or other biomolecule is not within the diffusion radius, the reactive intermediate is quenched by the surrounding aqueous or aqueous-based environment. As described further herein, the diffusion radius of the reactive intermediate can be tailored to specific microenvironment mapping considerations, and can be limited to the nanometer scale. In some embodiments, for example, the diffusion radius can be less than 100 nm, less than 50 nm, less than 20 nm, less than 10 nm or less than 5 nm, such as 1-5 nm. Moreover, in some embodiments, a protein labeling agent can be functionalized with a marker, such as biotin or luminescent markers for aiding in analysis.

As described herein, the tetrapyrrole photocatalyst includes a metal center that can be placed in an excited state for activating the protein labeling agent to a reactive intermediate via energy transfer. In some embodiments, the excited state of the tetrapyrrole photocatalyst can be quenched by a reductant, thereby returning the metal center to the ground state. The energy transfer to the reactive intermediate can subsequently occur from the ground state of the tetrapyrrole photocatalyst. In some embodiments, the tetrapyrrole photocatalyst absorbs electromagnetic radiation having wavelength longer than 600 nm or 650 nm to achieve an excited state. The tetrapyrrole photocatalyst, for example, may absorb radiation having a wavelength in the range of 650-1100 nm to achieve an excited state. Use of longer wavelength radiation can permit the radiation to penetrate tissue, thereby enabling the tetrapyrrole photocatalyst to interact with the radiation in a variety of in-vivo environments. Energy transfer from the catalyst to the protein labeling agent can occur via a variety of mechanisms described further herein, including Dexter energy transfer or single electron transfer. The energy transfer can occur from an excited state or ground state of the tetrapyrrole photocatalyst.

In some embodiments, the tetrapyrrole photocatalyst comprises a metal center. The metal center can comprise a transition metal or silicon, in some embodiments.

In another aspect, a composition for proximity-based labeling comprises a catalyst, and a protein labeling agent selected from the group consisting of thiatriazoles, sulfoximines, sulfilimines, anilines, acyl azides, ylides and dizo compounds. The catalyst has electronic structure to activate the protein labeling agent to a reactive intermediate via energy transfer. The reactive intermediate reacts or crosslinks with a protein or other biomolecule within the diffusion radius of the reactive intermediate, as described herein. The catalyst can comprise any catalyst operable to activate the protein labeling agent to the reactive intermediate. In some embodiments, the catalyst is a tetrapyrrole photocatalyst detailed herein.

In another aspect, conjugates for proximity-based labeling are described herein. A conjugate comprises a catalyst coupled to a biomolecular binding agent. The catalyst can have electronic structure for energy transfer to a protein labeling agent for generation of a reactive intermediate as described above. In some embodiments, the catalyst comprises a tetrapyrrole photocatalyst described herein. The biomolecular binding agent, in some embodiments, can be used to selectively locate or target the catalyst to a specific environment for mapping. The biomolecular binding agent, for example, locate the catalyst in the desired cellular environment for proximity labeling and associated analysis. As described herein, the cellular environment can be in vivo. The biomolecular binding agent can comprise a protein, polysaccharide, nucleic acid, or lipid, in some embodiments. In some instances, the biomolecular binding agent can comprise a multivalent display system comprising a protein, polysaccharide, nucleic acid, or lipid. Moreover, the biomolecular binding agent can also be a small molecule ligand with a specific binding affinity for a target protein.

In a further aspect, methods of proximity-based labeling are described herein. A method of proximity-based labeling comprises providing a catalyst, and activating a protein labeling agent to a reactive intermediate with the catalyst. The reactive intermediate couples or bonds to a protein. In some embodiments, the catalyst is coupled to a biomolecular binding agent to selectively locate or target the catalyst to a specific environment for protein mapping in conjunction with the protein labeling agent. The catalyst, conjugate, and protein labeling agent can have composition and/or properties described above, including tetrapyrrole photocatalysts, and in the following detailed description and Appendix attached hereto.

These and other embodiments are further described in the following detailed description.

### BRIEF DESCRIPTION OF THE DRAWINGS

FIGS. 1-6 illustrate various tetrapyrrole rings and associated tetrapyrrole photocatalysts comprising metal centers (M), according to some embodiments.

FIG. 7 illustrates various protein binding agents comprising azides, phenols, anilines, and thiatriazoles, according to some embodiments.

FIG. 8 illustrates various protein binding agents comprising acyl azides and diazirnines, according to some embodiments.

FIG. 9 illustrates various protein binding agents comprising sulfoximines, according to some embodiments.

FIG. 10 illustrates various protein binding agents comprising sulfilimines, according to some embodiments.

FIG. 11 illustrates various protein binding agents comprising ylides, according to some embodiments.

FIG. 12 illustrates various markers or affinity tags for protein labeling agents, according to some embodiments.

FIG. 13 illustrates various transition metal complexes for use in compositions, systems and methods herein for in vivo proximity-based labeling, according to some embodiments.

FIG. 14 illustrates various organic catalysts for use in compositions, systems and methods herein for in vivo proximity-based labeling, according to some embodiments.

FIG. 15 illustrates energy transfer between a tin (Sn) tetrapyrrole photocatalyst and protein labeling agent according to some embodiments.

FIG. 16 illustrates a synthetic pathway of a tin (Sn) tetrapyrrole photocatalyst according to some embodiments.

FIGS. 17A and 17B illustrate a synthetic pathway of a conjugate comprising tin (Sn) tetrapyrrole photocatalyst according to some embodiments.

FIG. 18 illustrates a synthetic pathway of a protein labeling agent functionalized with a marker according to some embodiments.

FIG. 19 illustrates conversion of azidobenzoic acid with a red light absorbing tetrapyrrole photocatalyst described herein according to some embodiments.

FIG. 20 provides conversion yields of azidobenzoic acid with a red light absorbing tetrapyrrole photocatalyst described herein in the presence and absence of various reductants according to some embodiments.

FIG. 21 is time-resolved absorption spectroscopy of Sn (OH)-chlorin e6 photocatalyst in the presence of phenyl azide or NADH.

FIG. 22A illustrates protein labeling with tetrapyrrole photocatalyst described herein according to some embodiments.

FIG. 22B provides Western blot results for protein biotinylation via tetrapyrrole photocatalyst labeling systems described herein, according to some embodiments.

FIG. 22C details photonic control over protein biotinylation. A labeling reaction was prepared, and aliquots were taken every 2 min. Samples were irradiated with red light for 2 min. at 4, 10 m and 16 min. time points.

FIG. 22D details proximity labeling through tissue with blue light and red light initiated photocatalytic labeling systems, according to some embodiments.

FIG. 23A illustrates primary anti-EGFR antibodies and red-light labeling conjugates comprising secondary antibodies for labeling microenvironments on living A549 cells according to some embodiments.

FIG. 23B provides STED microscopy of photolabeled cells with and without anti-EGFR primary antibodies. The inset represents a magnified region of interest illustrating radial labeling clusters overlaid with individual EGFR protein microenvironments. Depicted scale bar is 2 μm for no primary, 3 μm for anti-EGFR, and 1 μm for the zoomed inset.

FIG. 23C details a quantitative proteomic volcano plot of enriched proteins, according to some embodiments.

FIG. 24A illustrates a scheme for biotinylation of erythrocyte surfaces in whole blood with red-light initiated photocatalytic labeling systems described herein according to some embodiments.

FIG. 24B provides Western blot analysis of erythrocyte membrane lysate from isotype TER119-directed photolabeling.

FIG. 24C provides flow cytometry of isotype or TER119 photolabeled cells, according to some embodiments.

FIG. 24D is a quantitative proteomics volcano plot of identified proteins after whole blood labeling with red-light initiated photocatalytic labeling systems described herein according to some embodiments.

### DETAILED DESCRIPTION

Embodiments described herein can be understood more readily by reference to the following detailed description, examples, and Appendix and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, boron, oxygen and/or sulfur.

The term “heterocycle” as used herein, alone or in combination, refers to an mono- or multicyclic ring system in which one or more atoms of the ring system is an element other than carbon, such as boron, nitrogen, oxygen, and/or sulfur or phosphorus and wherein the ring system is optionally substituted with one or more ring substituents. The heterocyclic ring system may include aromatic and/or non-aromatic rings.

The term “alkoxy” as used herein, alone or in combination, refers to the moiety RO—, where R is alkyl, alkenyl, or aryl defined above.

The term “halo” as used herein, alone or in combination, refers to elements of Group VIIA of the Periodic Table (halogens). Depending on chemical environment, halo can be in a neutral or anionic state.

Terms not specifically defined herein are given their normal meaning in the art.

### I. Proximity-Based Labeling Compositions

In one aspect, compositions and methods are described herein for providing a microenvironment mapping platform operable to selectively identify various features, including in vivo protein-protein interactions on cellular membranes. In some embodiments, a composition comprises a tetrapyrrole photocatalyst, and a protein labeling agent, wherein the tetrapyrrole photocatalyst has electronic structure to activate the protein labeling agent to a reactive intermediate via energy transfer. As set forth herein, tetrapyrrole catalyst comprises a metal center participating in energy transfer to the protein labeling agent. In some embodiments, for example, the catalyst engages in Dexter energy transfer with the protein labeling agent. The energy transfer can proceed via single electron transfer, in some embodiments.

The energy transfer to the protein labeling agent can originate from an excited state of the tetrapyrrole photocatalyst electronic structure, in some embodiments. The excited state of the catalyst, for example, can be a singlet excited state or triplet excited state. The excited state of the tetrapyrrole photocatalyst can be generated by one or more mechanisms, including energy absorption by the photocatalyst. In some embodiments, the excited state is induced by absorption of one or more photons. In other embodiments, the catalyst may be placed in an excited state by interaction with one or more chemical species in the surrounding environment. Alternatively, energy transfer to the protein labeling agent, including electron transfer, may originate from a ground state of the catalyst electronic structure. The excited state of the tetrapyrrole photocatalyst can be quenched by reductant, returning the tetrapyrrole photocatalyst to the ground state. The energy transfer, including single electron transfer, can then proceed from the ground state of the tetrapyrrole photocatalyst to the protein labeling agent, resulting in the formation of the reactive intermediate.

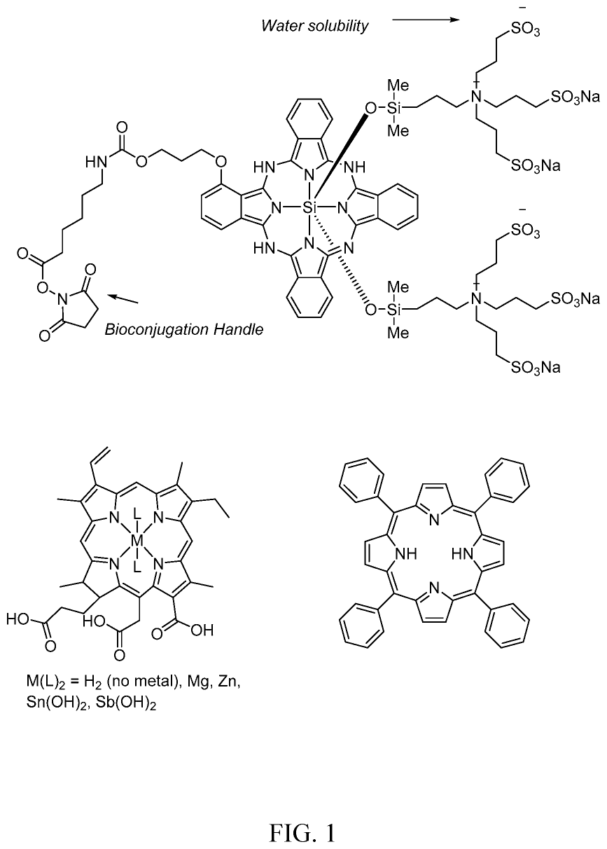

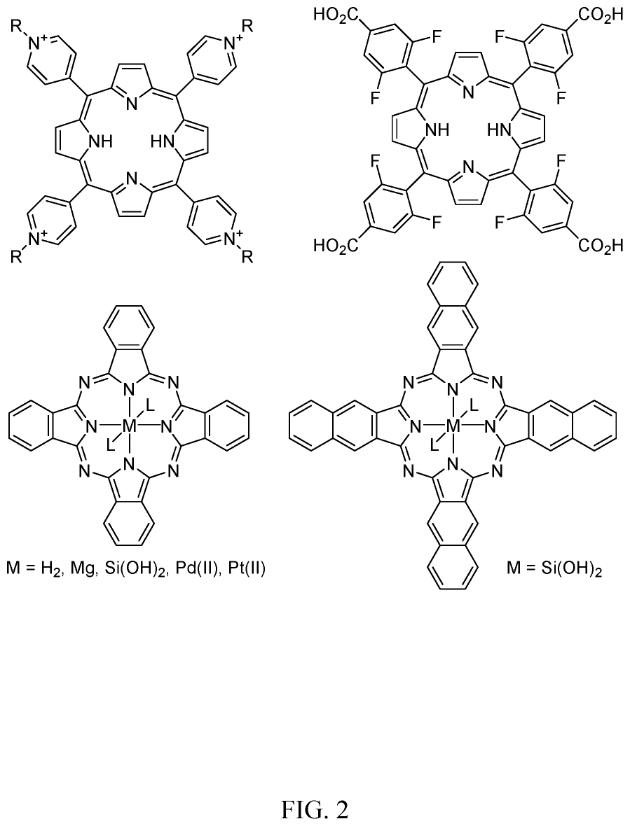

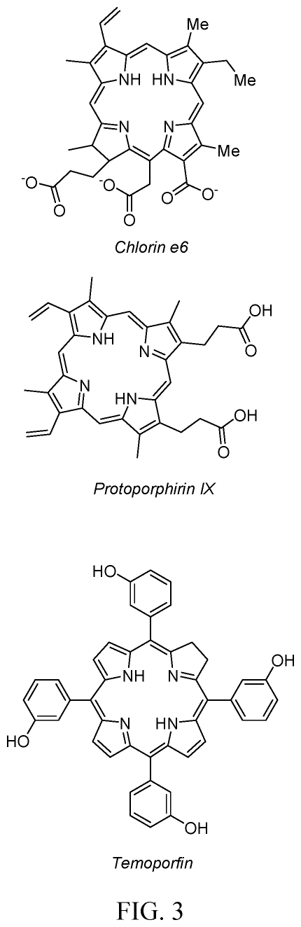

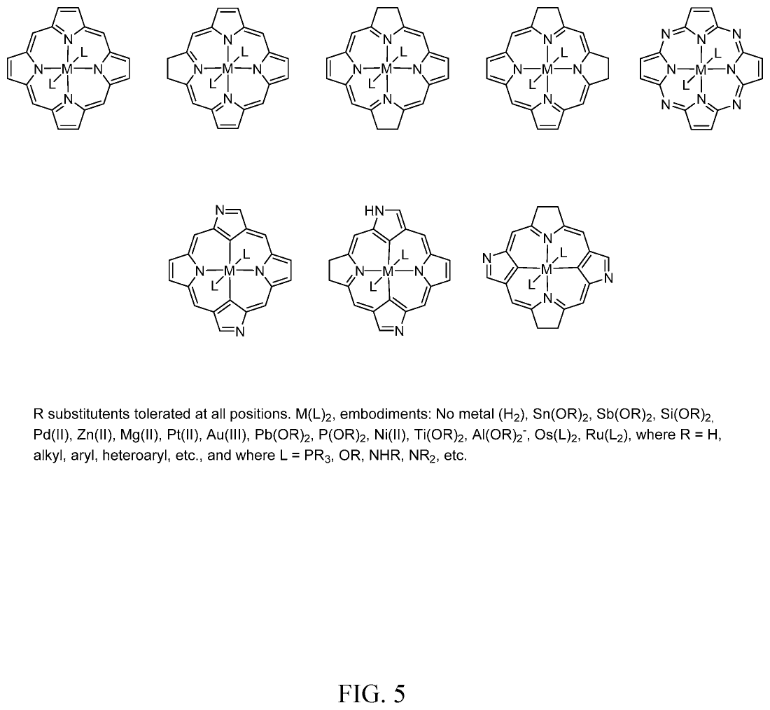

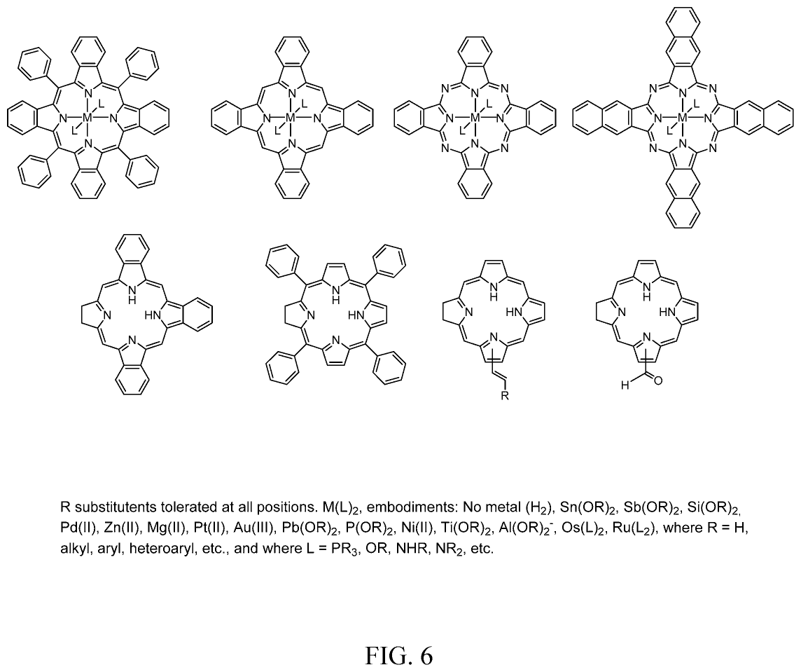

In some embodiments, the tetrapyrrole photocatalyst absorbs electromagnetic radiation having wavelength longer than 600 nm to achieve an excited state. The tetrapyrrole photocatalyst, for example, may absorb radiation having a wavelength in the range of 600-1100 nm to achieve an excited state. Use of longer wavelength radiation can permit the radiation to penetrate tissue, thereby enabling the tetrapyrrole photocatalyst to interact with the radiation in a variety of in vivo environments. As described herein, the tetrapyrrole photocatalyst comprises a metal center. The metal center can be a transition metal or silicon, in some embodiments. FIGS. 1-6 illustrate various tetrapyrrole rings and associated photocatalysts comprising metal centers, according to some embodiments described herein. Tetrapyrrole photocatalyst can include any metal center consistent with performing the energy transfer to a protein labeling agent. In some embodiments, the metal center is a transition metal, alkaline earth metal, or metalloid. Suitable transition metal can include noble metals or Groups 4-10 transition metals. In some embodiments, the metal center of the tetrapyrrole photocatalyst is selected from the group consisting of magnesium, zinc, tin, antimony, silicon, palladium, platinum, osmium, iridium, gold, lead, aluminum, phosphorus, and ruthenium.

The tetrapyrrole photocatalyst, in some embodiments, can be modified with one or more functionalities for altering solubility of the tetrapyrrole photocatalyst in various media. The tetrapyrrole photocatalyst, for example, can have one or more polar or ionizable functionalities on the pyrole or pyrole-like rings for enhancing solubility in water or aqueous-based cellular environments. In some embodiments, the tetrapyrrole photocatalyst have one or more carboxyl, hydroxyl, and/or amine functionalities. Alternatively, the tetrapyrrole photocatalyst can exhibit one or more hydrophobic constituents.

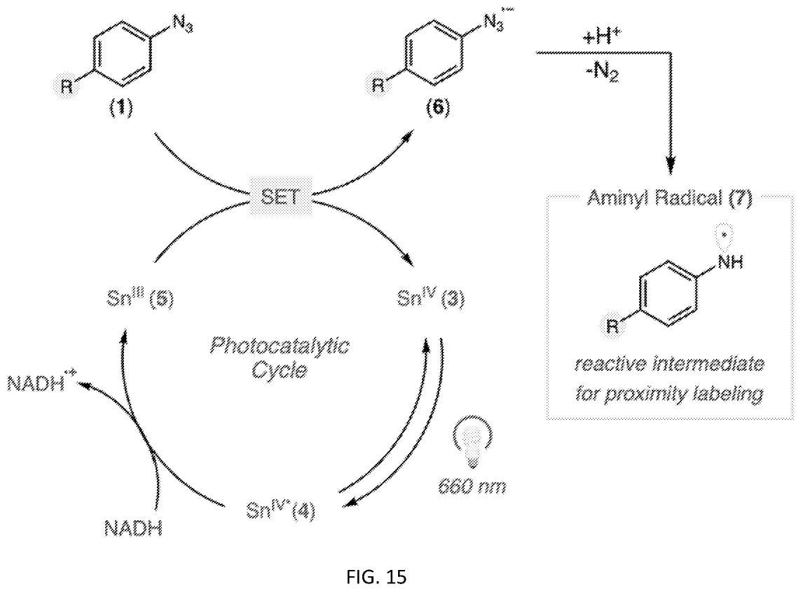

FIG. 15 illustrates energy transfer between a tin (Sn) tetrapyrrole photocatalyst and protein labeling agent according to some embodiments. As illustrated in FIG. 15, the tin tetrapyrrole photocatalyst (3) is placed in an excited state (4) via the absorption of electromagnetic radiation of 660 nm. A reductant (herein NADH) quenches the excited state of the tin tetrapyrrole photocatalyst, returning the catalyst to the ground state. The reduced tin tetrapyrrole photocatalyst (5) undergoes single electron transfer (SET) with the protein labeling agent, here an aryl azide (6), to form an aminyl radical (7) as the reactive intermediate. The single electron transfer regenerates the tin tetrapyrrole photocatalyst. Any reductant consistent with the technical objectives described herein may be used in conjunction with the tetrapyrrole photocatalyst to facilitate energy transfer from the photocatalyst ground state. As shown in FIG. 15, NADH is a suitable reductant. Additional reductants include glutathionone and ascorbate, in some embodiments. Specific identity of the reductant can be determined by the specific identity of the tetrapyrrole photocatalyst.

Energy transfer, including electron transfer, to the protein labeling agent forms a reactive intermediate of the protein labeling agent. The reactive intermediate reacts or crosslinks with a protein or other biomolecule within the diffusion radius of the reactive intermediate. If a protein or other biomolecule is not within the diffusion radius, the reactive intermediate is quenched by the surrounding environment, which may be an aqueous or aqueous-based environment. The diffusion radius of the reactive intermediate can be tailored to specific microenvironment mapping (proximity-based labeling) considerations, and can be limited to the nanometer scale. In some embodiments, for example, the diffusion radius of the reactive intermediate can be less than 100 nm, less than 50 nm, less than 10 nm, less than 5 nm, less than 4 nm, less than 3 nm, or less than 2 nm prior to quenching in the surrounding environment. The diffusion radius can be 0.5 nm to 10 nm, in some embodiments. Accordingly, the reactive intermediate will react or crosslink with a protein or other biomolecule within the diffusion radius or be quenched by the surrounding environment if no protein or biomolecule is present. In this way, high resolution of the local environment can be mapped via concerted effort between the catalyst and protein labeling agent. Additionally, the reactive intermediate can exhibit a t_{1/2} less than 5 ns, less than 4 ns, or less than 2 ns prior to quenching, in some embodiments. The reactive intermediate, for example, can exhibit a t_{1/2} less of 1-5 ns. In additional embodiments, the diffusion radius can be extended to between 5-500 nm though extension of the reactive intermediate half-life. For example, in some embodiments, the reactive intermediate can have a half-life of 1-100 μs, or greater.

In some embodiments, tetrapyrrole photocatalysts can be coupled to a biomolecular binding agent to provide a conjugate. The biomolecular binding agent, in some embodiments, can be used to selectively locate or target the catalyst to a specific environment for mapping. The biomolecular binding agent, for example, locate the catalyst in the desired cellular environment for proximity labeling and associated analysis. The biomolecular binding agent can comprise a protein, polysaccharide, nucleic acid, or lipid, in some embodiments. In some instances, the biomolecular binding agent can comprise a multivalent display system comprising a protein, polysaccharide, nucleic acid, or lipid. Moreover, the biomolecular binding agent can also be a small molecule ligand with a specific binding affinity for a target protein.

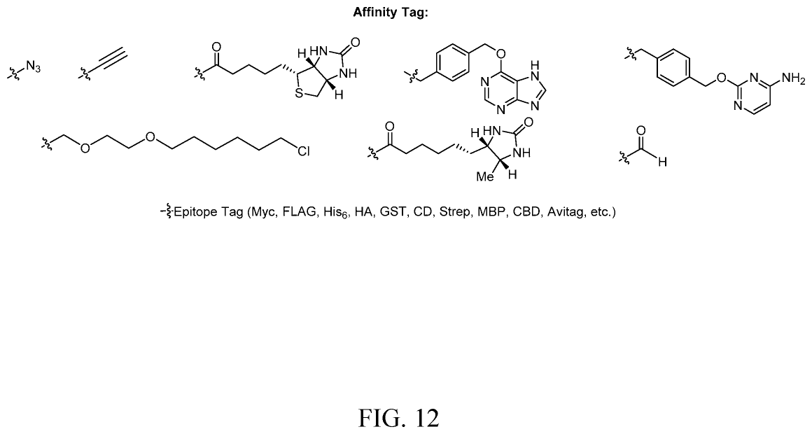

The protein labeling agent forming the reactive intermediate upon energy transfer from the tetrapyrrole photocatalyst, in some embodiments, can comprise an azide, diazirine, phenol, thiatriazole, sulfilimine, sulfoximine, ylide, diazo, aniline, or mixtures thereof. In some embodiments, the protein labeling agent can be functionalized with a marker, such as biotin. In some embodiments, the marker is desthiobiotin. The marker can assist in identification of proteins labeled by the protein labeling agent. The marker, for example, can be useful in assay results via western blot and/or other analytical techniques. Markers can include alkyne, azide, FLAG tag, fluorophore, and chloroalkane functionalities, in addition to biotin and desthiobiotin. FIG. 12 illustrates various markers or affinity tags, according to some embodiments.

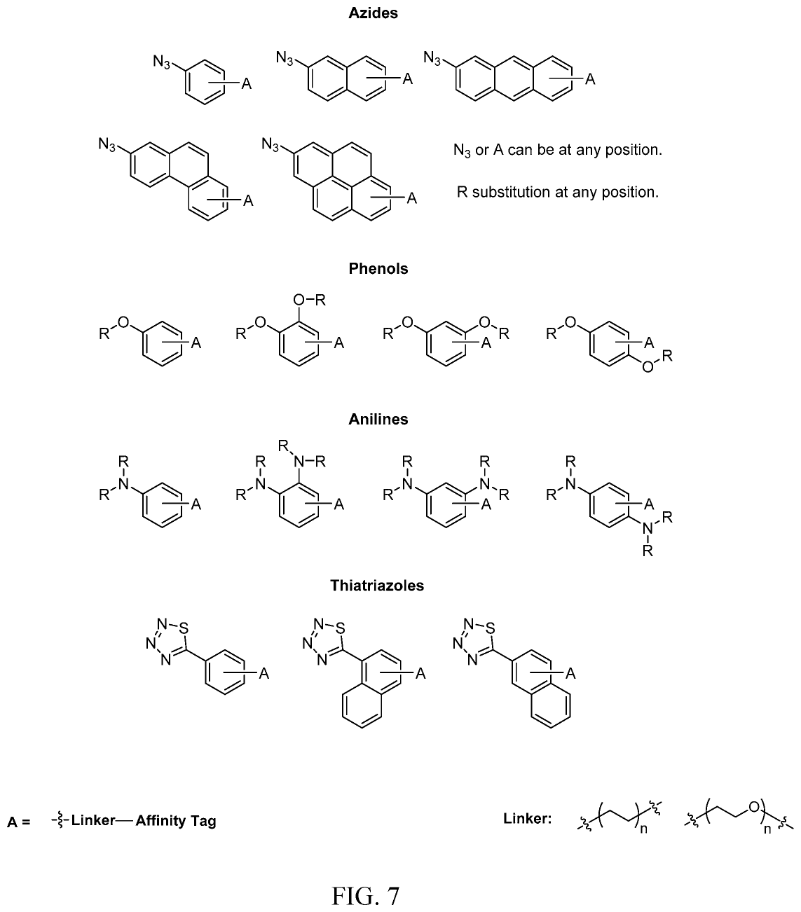

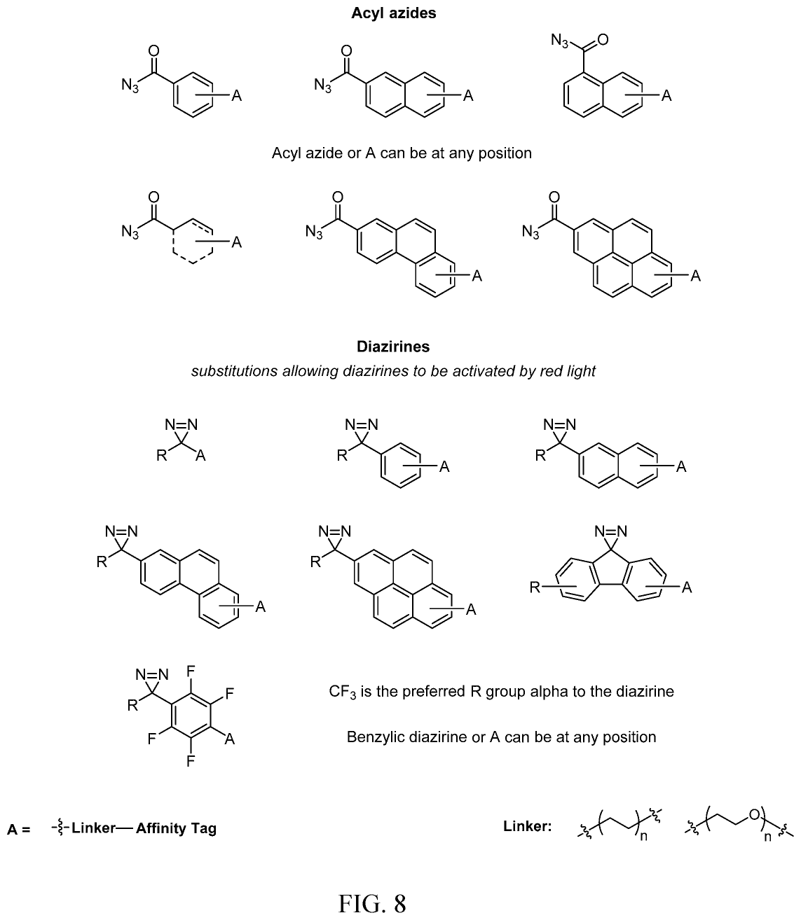

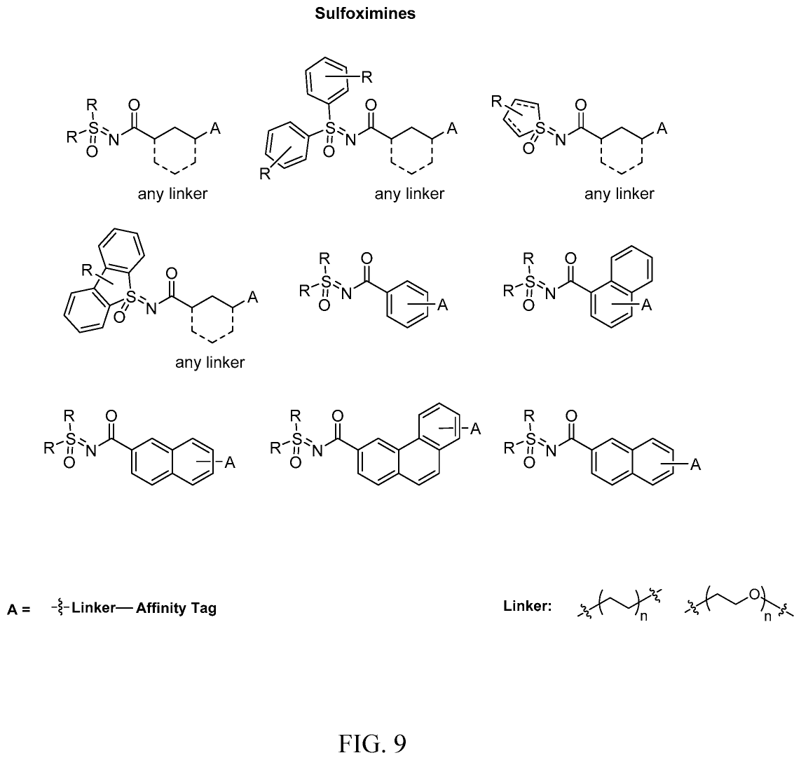

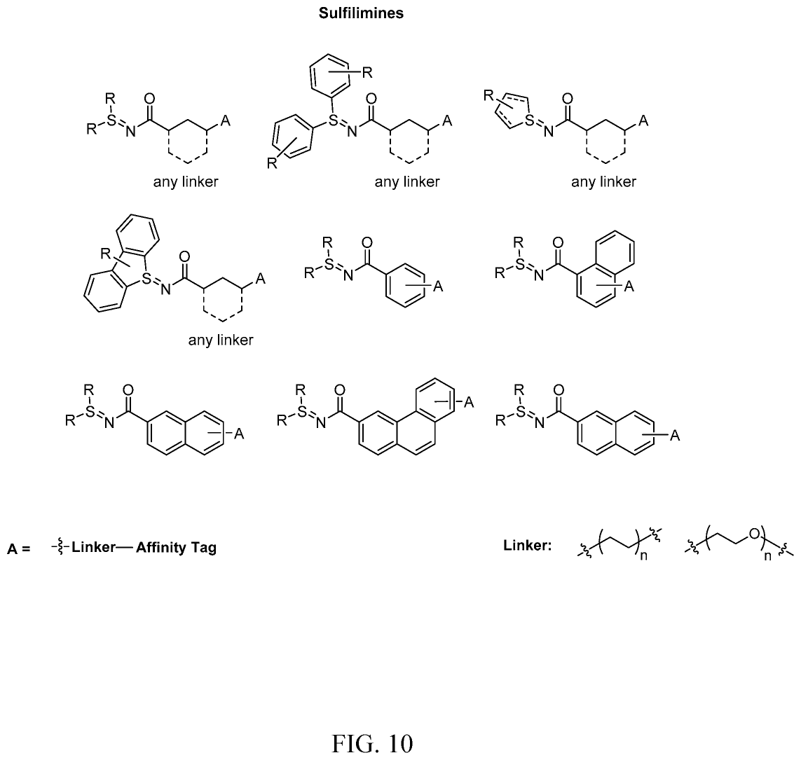

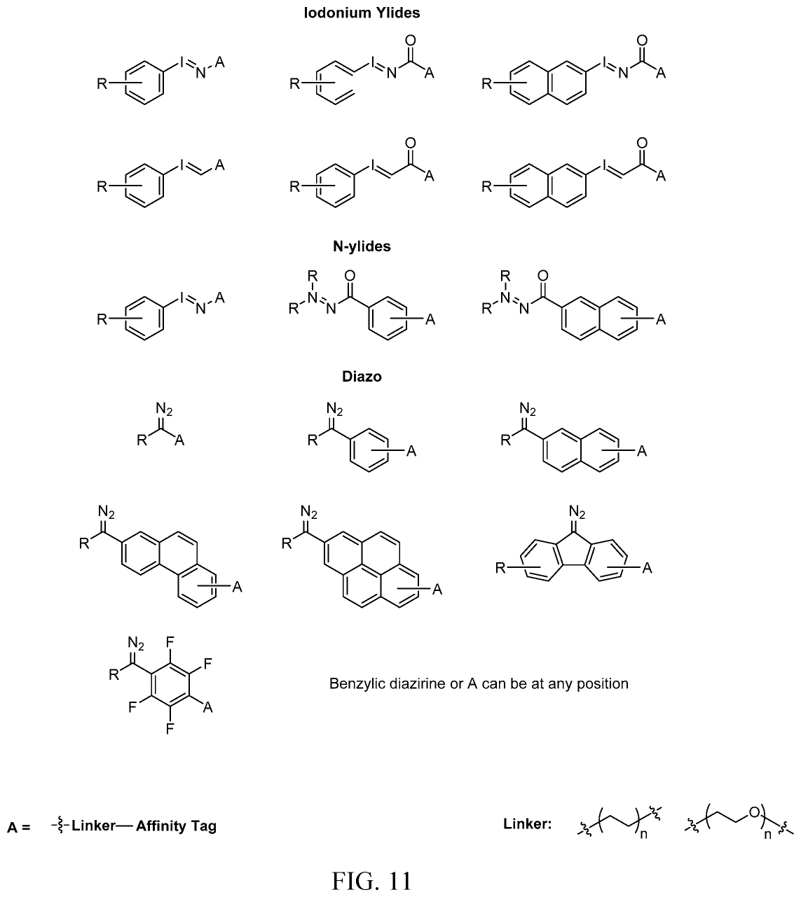

FIG. 7 illustrates various protein labeling agents comprising azides, phenols, anilines, and thiatriazoles, according to some embodiments. Azide protein binding agents, including aryl azides, can form reactive intermediates of nitrenes or aminyl radicals upon energy transfer from the tetrapyrrole photocatalyst. FIG. 8 illustrates various protein binding agents comprising acyl azides and diazirnines, according to some embodiments. FIG. 9 illustrates various protein binding agents comprising sulfoximines, according to some embodiments. FIG. 10 illustrates various protein binding agents comprising sulfilimines, according to some embodiments. FIG. 11 illustrates various protein binding agents comprising ylides, and diazo compounds, according to some embodiments.

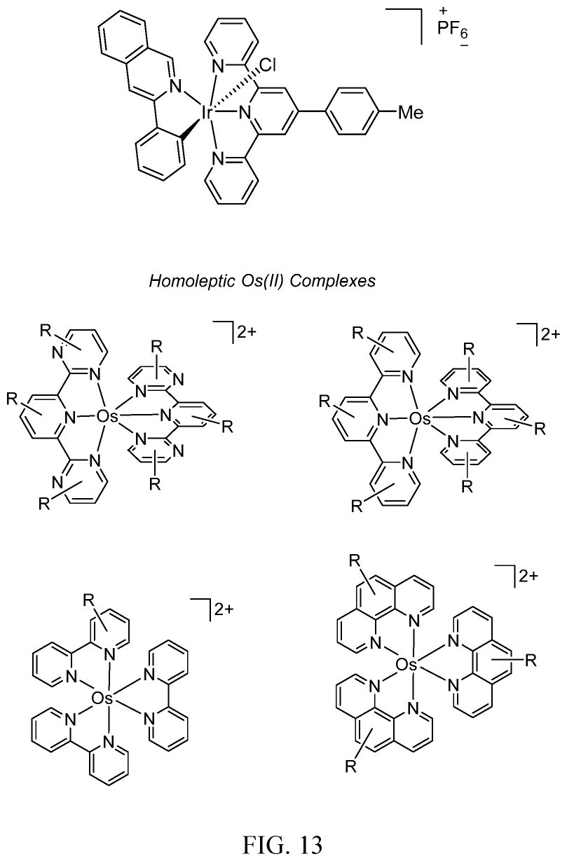

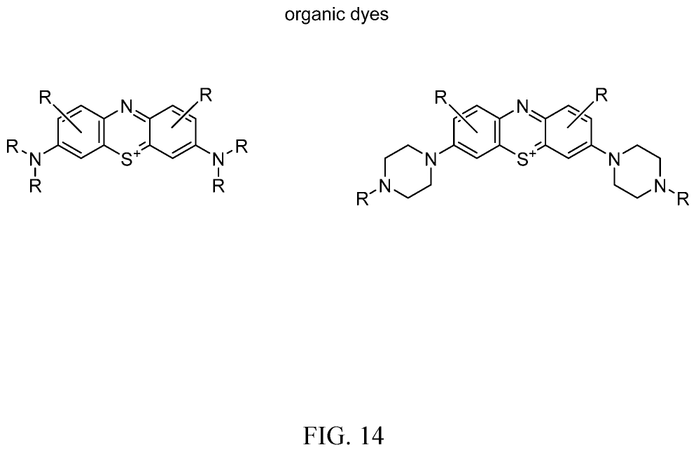

In some embodiments, the tetrapyrrole photocatalysts can be substituted by one or more differing transition metal catalysts for activating the protein labeling agent to the reactive intermediate via energy transfer. For example, a transition metal catalyst may comprise one or more tridentate ligands, such as terpyridine (terpy). FIG. 13 illustrates various transition metal complexes for use in compositions, systems and methods herein for in vivo proximity based labeling. Moreover, in some embodiments, tetrapyrrole photocatalysts can be substituted by one or more organic catalyst, such as organic dyes and/or other small molecules. FIG. 14 illustrates various organic catalysts for use in compositions, systems and methods herein for in vivo proximity based labeling.

As described above, in some embodiments, tetrapyrrole photocatalysts, transition metal catalysts, and/or organic catalysts can be functionalized with one or more moieties to enhance the hydrophilic character or hydrophobic character of the catalysts. In some embodiments, the catalysts are functionalized to render the catalysts soluble in aqueous or aqueous-based environments. Alternatively, the catalysts are functionalized to render the catalysts cell permeable.

In another aspect, a composition for proximity-based labeling comprises a catalyst, and a protein labeling agent selected from the group consisting of thiatriazoles, sulfoximines, sulfilimines, anilines, acyl azides, ylides and dizo compounds. The catalyst has electronic structure to activate the protein labeling agent to a reactive intermediate via energy transfer. The reactive intermediate reacts or crosslinks with a protein or other biomolecule within the diffusion radius of the reactive intermediate, as described herein. The catalyst can comprise any catalyst operable to activate the protein labeling agent to the reactive intermediate. In some embodiments, the catalyst is a photocatalyst detailed herein, including tetrapyrrole photocatalyst.

### II. Conjugates

In another aspect, conjugates for proximity-based labeling are described herein. A conjugate comprises a catalyst coupled to a biomolecular binding agent. The catalyst coupled to the biomolecular binding agent can comprise any catalyst described herein, including the tetrapyrrole photocatalysts, transition metal catalysts, and organocatalysts detailed in Section I above. Moreover, the biomolecular binding agent can comprise a protein, polysaccharide, nucleic acid, or lipid, in some embodiments. In some instances, the biomolecular binding agent can comprise a multivalent display system comprising a protein, polysaccharide, nucleic acid, or lipid. In some embodiments, the biomolecular binding agent can be a small molecule ligand with a specific binding affinity for a target protein. The biomolecular binding agent can be employed to locate the catalyst in the desired extracellular environment for proximity labeling and associated analysis. Accordingly, specific identity of the biomolecular binding agent can be selected according to the chemical and/or steric requirements of the desired target site for placement of the catalyst in the proximity based labeling process. Any biomolecular target site can be chosen, and target sites are not limited in the present disclosure. In some embodiments, target sites can be proteins for studying protein-protein interactions, including interaction with cellular membrane receptors. In some embodiments, for example, the biomolecular binding agent is an antibody, such as a secondary antibody for interacting with a primary antibody bound to the desired antigen. In other embodiments, the biomolecular binding agent is a ligand with specificity for a protein receptor of the cellular membrane, such as epidermal growth factor receptor (EGFR) or G protein-coupled receptor.

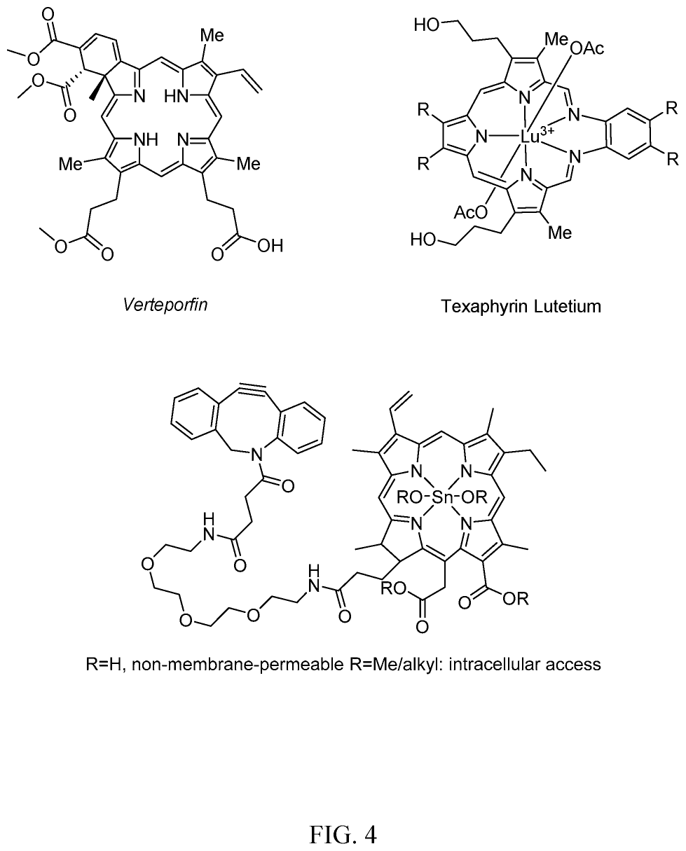

The biomolecular binding agent can be bonded to the catalyst. In some embodiments, the catalyst comprises a reactive handle or functionality for coupling the biomolecular binding agent. In some embodiments, for example, a catalyst can comprise one or more click chemistry moieties including, but not limited to, BCN, DBCO, TCO, tetrazine, alkyne, and azide. FIG. 4 illustrates various transition metal photocatalysts of Formula (I) having a reactive functionality for coupling a biomolecular binding agent.

### III. Systems for Proximity Based Labeling

In another aspect, systems for proximity-based labeling are described herein. A system, for example, comprises a conjugate including a catalyst coupled to a biomolecular binding agent, and a protein labeling agent activated by the catalyst for binding to a protein. The conjugate can comprise any catalyst and biomolecular binding agent described herein, including the embodiments detailed in Section II above, and the associated including the tetrapyrrole photocatalysts, transition metal catalysts, and organocatalysts detailed herein. The catalyst, for example, can have electronic structure to activate the protein labeling agent to a reactive intermediate via energy transfer. Moreover, the protein labeling agent can comprise any of the labeling agents described herein, including the protein labeling agents set forth in Section I above. Specific identity of the conjugate and associated protein labeling agent can be selected according to several considerations, such as the chemical nature and/or steric requirements of the biological environment to be mapped with the proximity-based labeling system.

Systems for proximity-based labeling described herein can be employed in various applications. In some embodiments, the systems enable target identification, wherein the conjugate and associated protein labeling agent permit identification of one or more molecules in a biological context by proteomics. Additionally, systems comprising the conjugate and protein labeling agent facilitate interactome mapping. Targeting a conjugate and protein labeling agent allows detection and identification of one or more molecules and neighboring interactors in a biological context by proteomics. Identification of such molecules by systems described herein can permit enrichment and/or purification of such molecules and neighboring interactors.

Additionally, systems comprising a conjugate and protein labeling agent further enable detection and identification of one or more molecules in a biological context via microscopy.

### IV. Methods of Proximity-Based Labeling

In another aspect, methods of proximity-based labeling are described herein. A method of proximity-based labeling comprises providing a conjugate comprising a catalyst coupled to a biomolecular binding agent, activating a protein labeling agent to a reactive intermediate with the catalyst, and coupling the reactive intermediate to a protein. The conjugate can comprise any catalyst, including tetrapyrrole photocatalyst, and biomolecular binding agent described herein, including the embodiments detailed in Section II above. Moreover, the protein labeling agent can comprise any of the labeling agents described herein, including the protein labeling agents set forth in Section I above. The protein labeling agent forming the reactive intermediate upon energy transfer from the tetrapyrrole photocatalyst, in some embodiments, can comprise an azide, diazirine, phenol, thiatriazole, sulfilimine, sulfoximine, ylide, diazo, aniline, or mixtures thereof. Specific identity of the conjugate and associated protein labeling agent can be selected according to several considerations, such as the chemical nature and/or steric requirements of the biological environment to be mapped with the proximity-based labeling system. In some embodiments of proximity-based labeling, the catalyst can be provided in the absence of a biomolecular binding agent.

Methods described herein can be employed to map various in vivo biological environments, including local areas of cellular membranes and/or the local extracellular environment. As described herein, the ability of tetrapyrrole photocatalysts to be activated with electromagnetic radiation greater than 600 nm enables mapping of in vivo biological environments well below tissue exteriors, such as the skin. In some embodiments, environments can be mapped at tissue depths of greater than 5 mm or greater than 10 mm. For example, in vivo mapping can occur at tissue depths of 5 mm to 50 cm, in some embodiments.

The conjugate comprising the catalyst and biomolecular binding agent may be targeted to a specific local region of a cellular membrane, such as a receptor of interest. Activation of the protein labeling agent can identify protein(s) and/or other molecules in the targeted local region. Notably, the activated protein labeling agent can also identify or label molecules associated with another cell in contact with the targeted cellular region. Therefore, intercellular interactions and intercellular environments can be elucidated and mapped with systems and methods described herein. The foregoing methods enable interactome mapping, and the identification of one or more molecules and neighboring interactors in a biological context by proteomics. Identification of such molecules by methods described herein can permit enrichment and/or purification of such molecules and neighboring interactors.

In some embodiments, multiple photocatalysts can be employed in proximity-based labeling systems and methods described herein. The photocatalysts can exhibit differing absorption profiles, thereby enabling selective proximity-based labeling dependent on the wavelength of excitation radiation provided. In some embodiments, photocatalysts and associated protein labeling agents described in Patent Cooperation Treaty Application Serial Number PCT/US2020/036285 can be used with photocatalysts and protein labeling agents described herein. Light having wavelength of 375-450 nm, for example, can be used to effectuate proximity-based labeling with the photocatalysts and protein labeling agents described in PCT/US2020/036285. Moreover, light having wavelength of 650-1100 nm can be used to effectuate proximity-based labeling in some embodiments described herein with tetrapyrrole photocatalysts and conjugates described in Sections I and II above. The differing photocatalysts can have different biomolecular binding agents to target differing cellular environments. Differing protein labeling agents between the photocatalysts may also be used. Under this analytical regime, many local cellular environments may be mapped, thereby elucidating previously unknown biomolecular interactions and relationships.

These and other embodiments are further illustrate by the following non-limiting examples.

### EXAMPLE 1

### Synthesis of Tetrapyrrole Photocatalyst

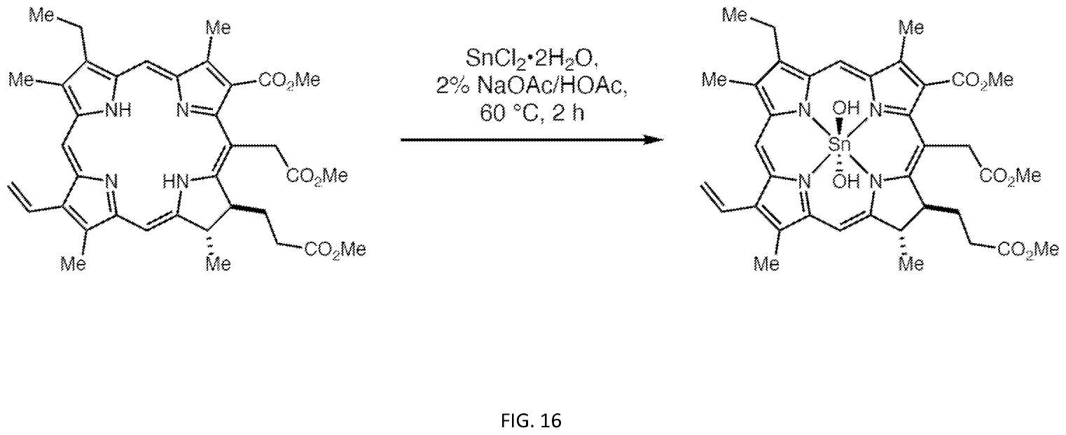

Tin (Sn) metalated chlorin e6 photocatalyst was synthesized according to the reaction scheme of FIG. 16. Chlorin e6 trimethylester (7.6 mg, 0.12 mmol) and tin chloride dihydrate (26.8 mg, 0.12 mmol) were added to an 8 ml vial equipped with a magnetic stir bar and dissolved in a 2% NaOAc/glacial acetic solution (0.03 M). This solution was then heated to 60° C. and stirred for 2 hours. The mixture was then let to cool to room temperature, diluted with 10 ml of 1N HCl, and extracted three times with 200 ml DCM. The combined extracts were dried over sodium sulfate, filtered, and concentrated under reduced pressure to yield compound S1 as a dark blue solid (3.6 mg, 38.3% yield).

### EXAMPLE 2

### Synthesis of Tetrapyrrole Photocatalyst

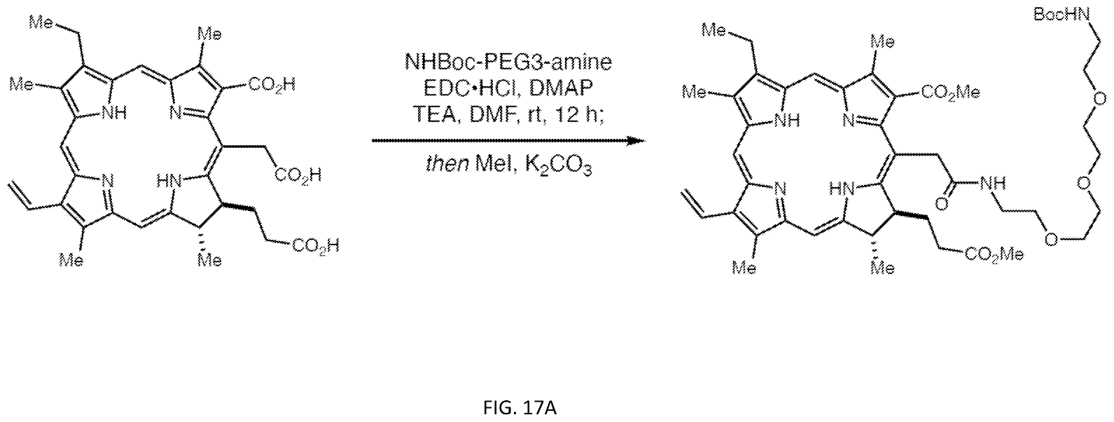

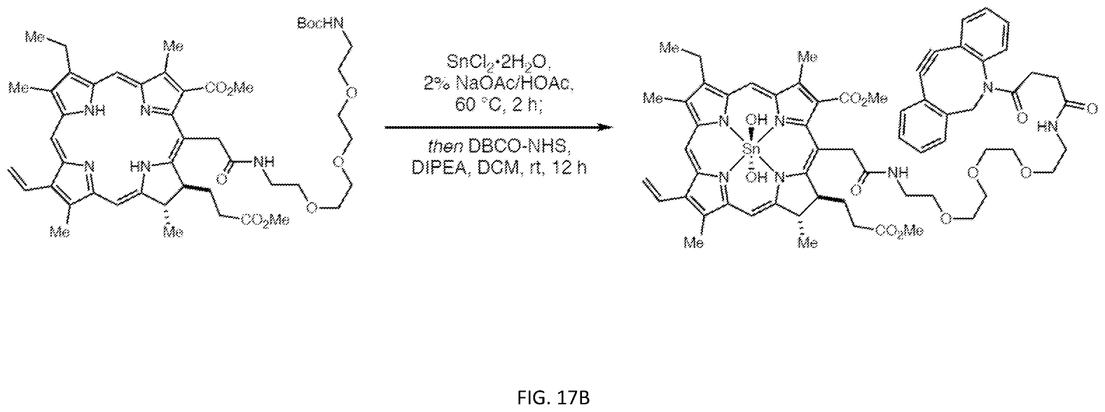

Chlorin e6-PEG3-NHBoc (S2) was synthesized according to the reaction scheme of FIG. 17A. Chlorin e6 (100 mg, 0.15 mmol, Cayman Chemicals, cat. 21684), EDC·HCl (20.8 mg, 0.11 mmol), triethylamine (42.5 μl, 0.31 mmol), and DMAP (1.2 mg, 0.012 mmol) were added to a 40 ml vial equipped with a magnetic stir bar and then dissolved in DMF (0.2 M). After 10 minutes, t-Boc-Namido-PEG3-amine (68.3 mg, 0.23 mmol, BroadPharm, cat. BP-20583) was added and the mixture was let to stir at room temperature for 12 hours. Following this, potassium carbonate (20 mg, 2 equiv) and methyl iodide (67.8 μl, 1.09 mmol) were added, and the resulting mixture was allowed to stir at room temperature for 1 hr. Following reaction completion, 1 ml of DMSO was added, and the resultant solution was then directly purified via prep-HPLC (10-100% ACN/H₂O w/0.1% formic acid) to afford compound S2 as a dark green oil (21.2 mg, 15.2% yield). Selective condensation of NHBoc-PEG3-amine onto the ethanoic (C34) carboxylic acid was confirmed by 2D NMR correlations and is consistent with previously observed regioselectivity.

Chlorin e6 Sn (OH) DBCO (S3) was synthesized according to the reaction mechanism of FIG. 17B. S2 (14.0 mg, 0.015 mmol) and tin chloride dihydrate (35.1 mg, 0.15 mmol) were added to an 8 ml vial equipped with a magnetic stir bar and dissolved in a 2% NaOAc/glacial acetic solution (0.03M). This solution was then heated to 60° C. and let to stir for 2 hours before the addition of a 50 μl of concentrated HCl. After one hour, the reaction was allowed to cool to room temperature and then diluted with DCM (3 mM). Then, the vial was sparged with N₂ for 15 minutes and cooled to 0° C. before the addition of DIPEA (500 μl, 29.0 mmol) and DBCO-NHS (25.0 mg, 0.062 mmol, BroadPharm). The reaction was stirred for 12 h at rt in the dark under inert atmosphere. The reaction was then concentrated under reduced pressure, taken up in the minimum amount of DMSO, and directly purified via prep-HPLC (10-100% ACN/H₂O w/0.1% ammonium hydroxide) to afford the tetrapyrrole photocatalyst as a dark blue film (6.8 mg, 35.3% yield).

### EXAMPLE 3

### Protein Labeling Agent Functionalized With Marker

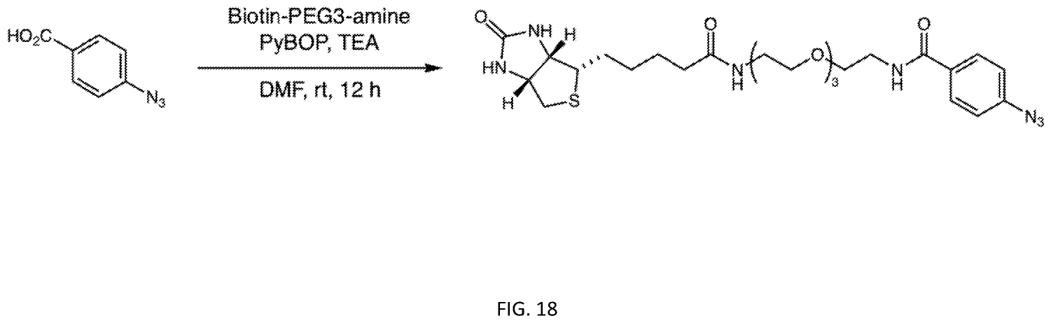

Biotin-PEG3-phenyl azide was synthesized according to the reaction scheme of FIG. 18. 4-azidobenzoic acid (50.0 mg, 0.30 mmol), PyBOP (224 mg, 0.43 mmol, 1.1 equiv.), and triethylamine (83.7 μl, 0.61 mmol, 2 equiv.) were added to an 8 ml vial and dissolved in a 0.5 ml of DMF. The reaction mixture was stirred at room temperature for 20 minutes before the addition of Biotin-PEG3-amine (128 mg, 0.30 mmol, 1 equiv.). This resultant solution was then allowed to stir at room temperature for 12 hours, after which the crude mixture was concentrated under reduced pressure, redissolved in DMSO, passed through a syringe filter (13 mm, 0.2 μm, PTFE, cat: 9720002) and directly purified via prep-HPLC (10-100% ACN/H₂O w/0.1% formic acid) to afford compound as a light brown wax (83.0 mg, 48.1% yield).

### EXAMPLE 4

### Red Light Tetrapyrrole Photocatalyst Activity

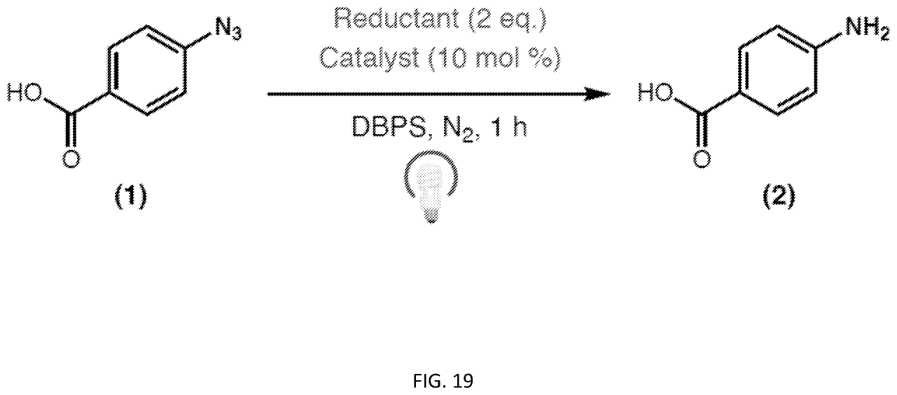

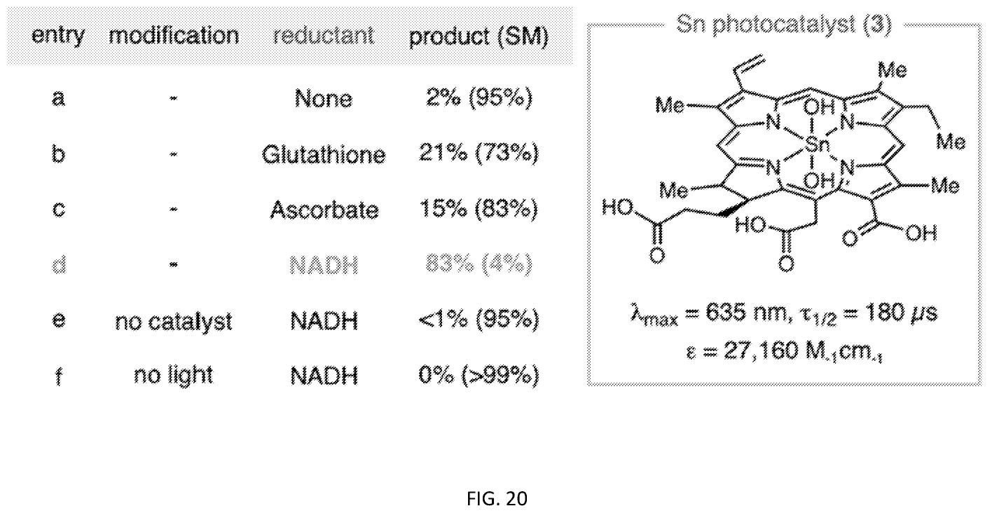

Several red light photocatalysts of varying redox properties were tested for the conversion of 4-azidobenzoic acid, as set forth in FIG. 19. Using a Sn-metalated chlorin e6 catalyst (3), trace conversion (5%) and small quantities of aniline product 2 (2%) were observed (FIG. 20, entry a). An addition of stoichiometric reductants, including glutathione, sodium ascorbate, or NADH led to dramatic yield improvements (FIG. 20, entries b-d), with NADH as the most effective (83% yield).

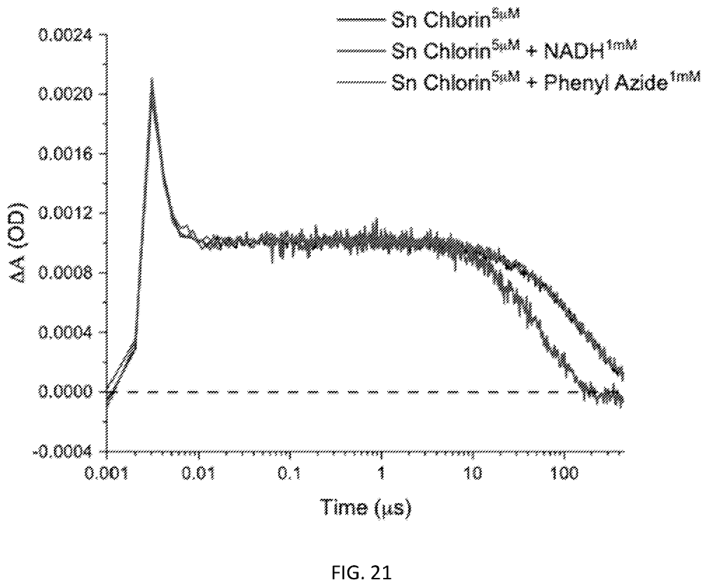

From these data, a mechanistic pathway was proposed, the pathway initiating via reductive quenching of the excited-state photocatalyst (4) with NADH to form a highly reducing organic ground state (E_{1/2}=−0.69 V vs Ag/AgCl), as illustrated in FIG. 15. This reduced species is poised to undergo single electron transfer (SET) to the aryl azide, 1 (E_{p/2}=−0.61 V vs Ag/AgCl), thereby regenerating the catalyst (3) (FIG. 15). Mesolytic cleavage of the azide radical anion (6) releases molecular nitrogen, and rapid protonation reveals an aminyl radical species (7) as a reactive intermediate for proximity labeling. Ultrafast transient-absorption spectroscopy revealed that the excited Sn-chlorin catalyst is quenched by NADH and not by aryl azide 1, providing support for the proposed ground state reductive electron transfer mechanism (FIG. 21).

Furthermore, electrochemical reduction of the Sn-chlorin e6 catalyst generated a species with significant spectral overlap with the transient-absorption signal of the photoexcited catalyst in the presence of NADH, supporting the generation of the reduced ground state catalyst.

### EXAMPLE 5

### In Vitro Labeling With Tetrapyrrole Photocatalytic Systems

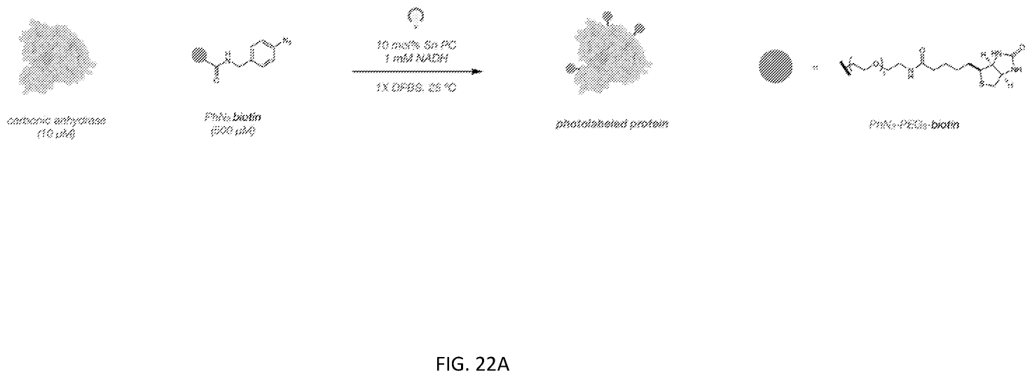

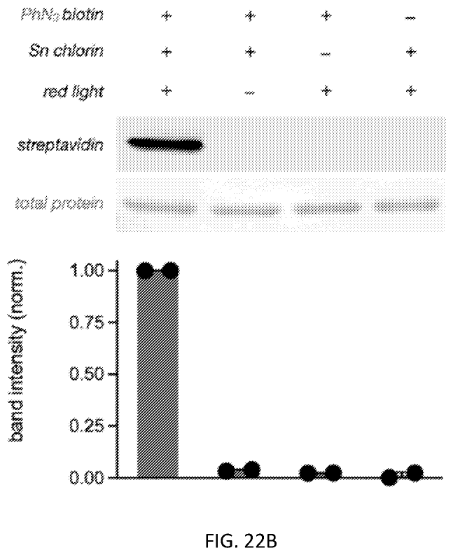

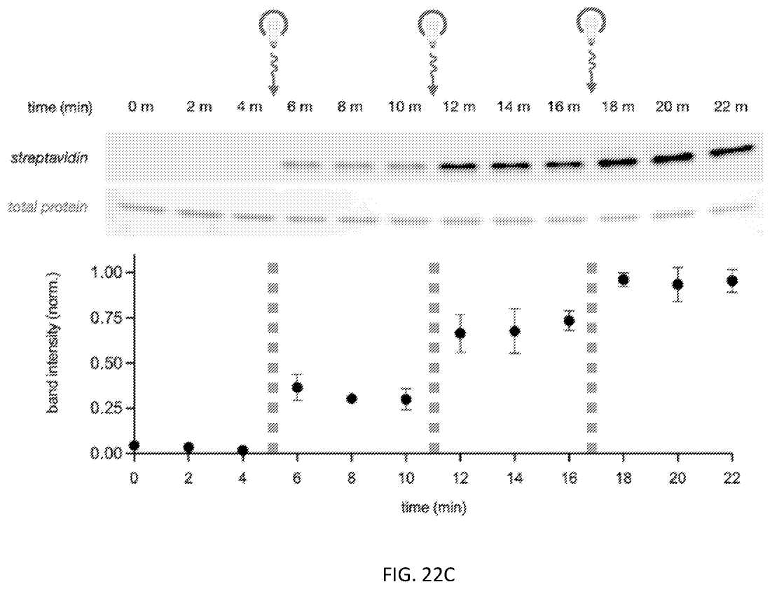

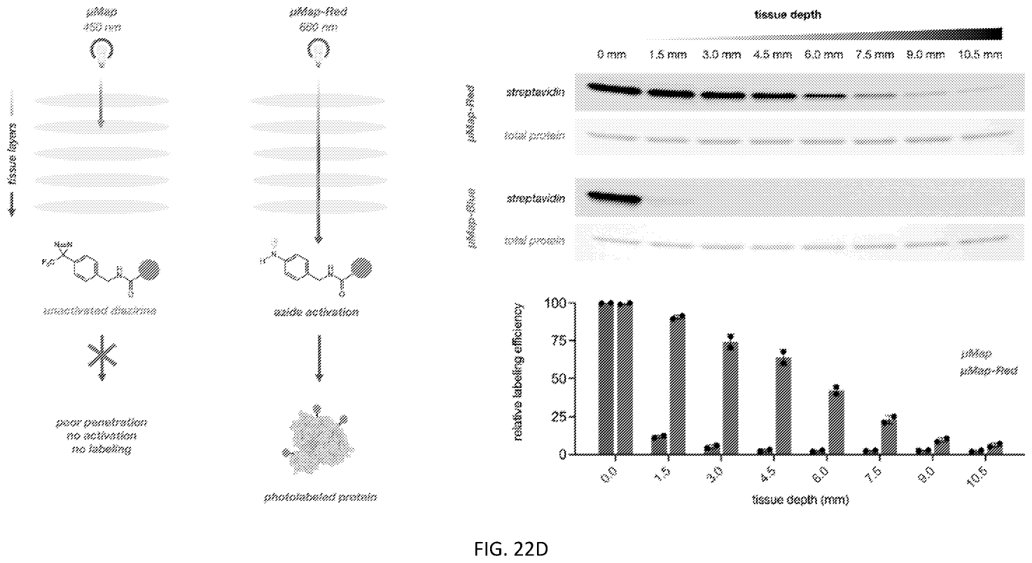

Tetrapyrrole photocatalyst activity in vitro was established by covalently tagging a recombinant protein in aqueous solution. Carbonic anhydrase was subjected to labeling (10 mol. % tetrapyrrole photocatalyst, 1 mM NADH, 500 μM PhN₃-biotin) as set forth in FIG. 22A. Robust protein biotinylation was observed (FIG. 22B). No labeling was observed in the absence of photocatalyst, PhN₃ probe, or light, and labeling intensity was commensurate with increasing irradiation times. Additionally, light dependence on labeling was observed with discrete increases in biotinylation following 2 min pulses of light (FIG. 22C). Given the long-term goal of applying this technology for proximity labeling in vivo, the efficiency of the present labeling system through increasing layers of tissue between the light source and the sample was probed (FIG. 22D). Both blue-light activated photocatalyst and red-light activated photocatalyst protocols achieved robust biotinylation in the absence of tissue obstruction, yet a sharp decrease (˜90%) in biotinylation efficiency of blue-light activated photocatalyst was observed with just 1.5 mm of tissue obscuring the light source, confirming poor penetration of blue light through dermal layers. Conversely, the red-light activated tetrapyrrole photocatalyst exhibited detectable labeling through increasing amounts of tissue (>10 mm) (FIG. 22D).

### EXAMPLE 6

### Cellular Labeling With Tetrapyrrole Photocatalytic Systems

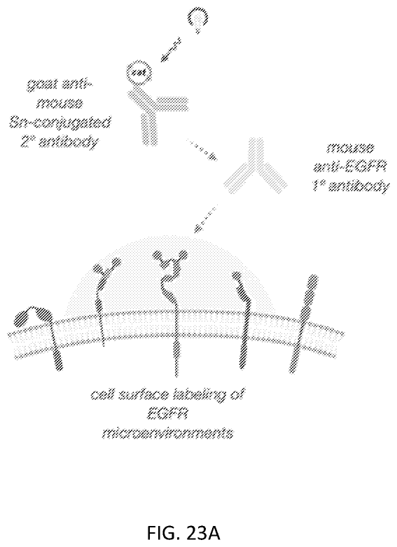

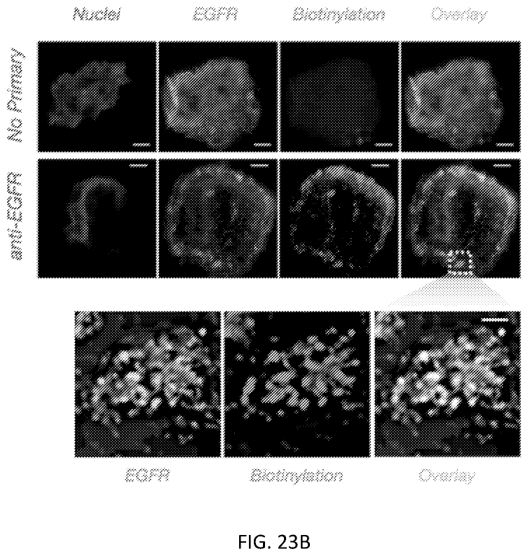

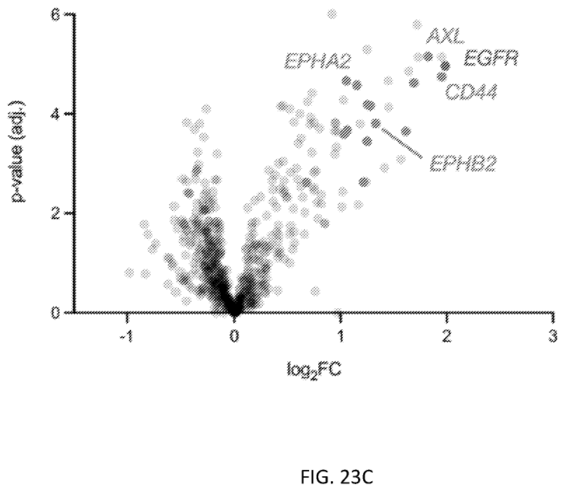

With a system for red-light activated labeling based on tetrapyrrole photocatalyst established, cellular labeling via the red-light activation was examined. As a model system, epidermal growth factor receptor (EGFR), a cell surface receptor-tyrosine kinase, was selected. Secondary antibodies conjugated to Sn-chlorin photocatalyst were synthesized, which could then be directed with primary antibodies to EGFR, as illustrated in FIG. 23A.

A549 cells were subjected to immunotargeted photolabeling (1 mM NADH, 500 μM PhN₃-biotin, 30 min irradiation) in the presence or absence of anti-EGFR antibodies. Spatially selective biotinylation was assessed via stimulated emission depletion (STED) super-resolution microscopy. As illustrated in FIG. 23B, Sn-chlorin photocatalytic labeling exhibited robust cell-surface biotinylation only in the presence of anti-EGFR antibodies, indicating low nonspecific binding or off-target labeling. The high resolution offered by STED microscopy also allowed qualitative assessment of colocalization of EGFR and labeling. As shown in FIG. 23B, a biotinylation signal was observed that strongly overlaid with EGFR staining, signifying confinement of labeling to the EGFR microenvironment.

To assess spatial selectivity of labeling, a measurement of the full width at half-maximum (fwhm) of biotinylation clusters estimated the Gaussian distribution oflabeling events to be 87±33 nm (n=50 clusters). This distribution is in agreement with the known longer lifetime of the aminyl radical intermediate (˜50 μs) with respect to carbenes (˜2 ns) generated using blue-light photocatalytic species, but nonetheless affords red-light proximity labeling systems described herein with the ability to profile nanoscale events in individual protein microenvironments.

Membrane lysate fractions from photolabeled cells were then generated and subjected to streptavidin enrichment and quantitative proteomics. Consistent with the STED analysis, EGFR enrichment was observed via Western blot only in samples that had been exposed to anti-EGFR antibody. Here, quantitative tandem mass tag (TMT) proteomics revealed 29 enriched proteins with log₂(FC)>1 (FIG. 23C). Satisfyingly, EGFR was the most enriched protein in the data set. Of these enriched proteins, 12 have previously validated physical interactions with EGFR (FIG. 23C), including CD44, a transmembrane glycoprotein known to regulate EGFR autophosphorylation. Additionally, one of the most enriched proteins, AXL, is a known substrate of EGFR phosphorylation. EPHA2 and EPHB2, also receptor proteintyrosine kinases, were highly enriched in the data set and are known to modulate vesicular trafficking of EGFR. Together, these data validate the accuracy of red-light absorbing tetrapyrrole photocatalytic systems described herein as a proximity labeling platform for profiling spatial connections in signaling pathways.

### EXAMPLE 7

### Cellular Labeling With Tetrapyrrole Photocatalytic Systems

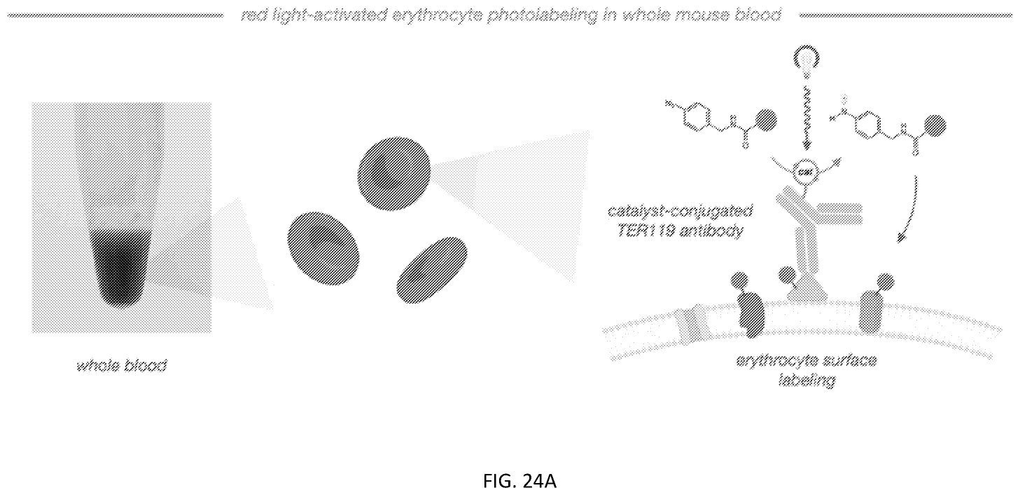

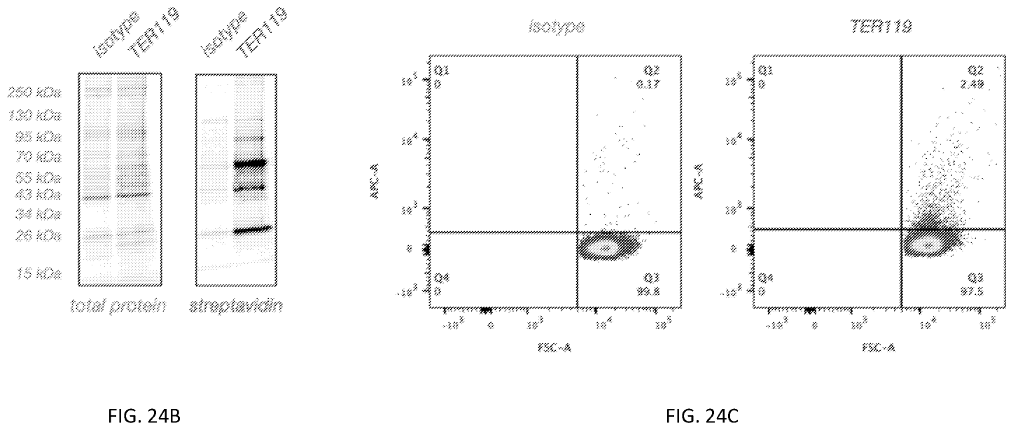

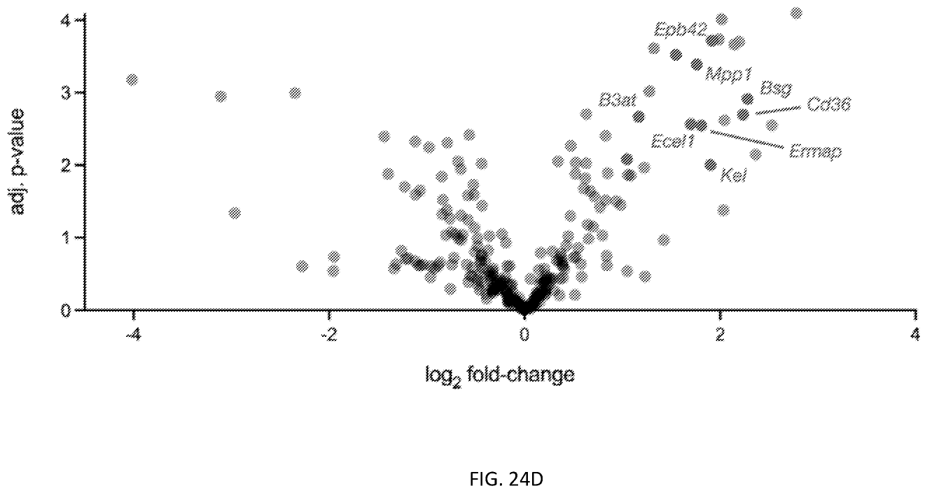

It was next sought to evaluate tetrapyrrole photocatalytic labeling systems described herein in a complex setting where blue light activation would not be feasible. Along these lines, whole blood presents high levels of biochemical complexity, and it was questioned whether tetrapyrrole photocatalytic labeling systems could be used in this setting to achieve selective proximity labeling. TER119, a well-characterized antibody raised against mature erythrocytes, was selected as the targeting modality for cell-surface labeling (FIG. 24A). Interestingly, although TER119 is monoclonal, it has been shown to bind several targets on red blood cells and remains a gold standard erythrocyte marker for flow cytometry analysis of whole blood.

First, we conjugated Sn-chlorin catalysts to both TER119 and a nontargeting isotype conjugated TER119, and minimal signal was observed with isotype conjugates (FIG. 24B). Intensity of labeling was proportional to TER119 concentration while no biotinylation was observed with any amount of isotype (FIG. 24B). Remarkably, this experiment was repeated using blue-lightactivated proximity labeling photocatalyst, and no signal was observed across all conditions, consistent with the observed poor tissue penetration of blue light (FIG. 22D). In parallel, photolabeled blood was analyzed via flow cytometry by staining with fluorescent Neutravidin-DyLight 650. As shown in FIG. 24C, minimal signal was observed in the isotype reactions, while a significant (˜15-fold) increase in biotinylated cells was seen with TER119 reactions, indicating erythrocyte labeling in whole blood. Finally, quantitative proteomics was performed on enriched membrane lysates (FIG. 24D). 24 proteins were observed that were strongly enriched [log₂(FC)>1] in the data set, the majority of which are known erythrocyte cell surface proteins (FIG. 24D). In particular, basigin (Bsg), Cd36, Kel, erythrocyte membrane associated protein (Ermap), 55 kDa erythrocyte membrane protein (Mpp1), band 3 anion transport protein (B3at), and protein 4.2 (Epb42) were among the most enriched. These targets constitute the major mouse erythrocyte membrane proteins and blood antigen group glycoproteins and likely represent the major TER119 antigen ensemble. Additionally, enrichment of several cytoskeletal proteins was observed (FIG. 24D), including spectrins alpha and beta as well as ankyrin and alpha-adducin.

In view of the foregoing, a red-light-activated proximity labeling platform has been developed based tetrapyrrole photocatalyst, protein labeling agents, and conjugates described herein. This system exhibits phototonic and spatiotemporal control over labeling and can operate in both simple and complex biological environments.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A composition for proximity-based labeling comprising:
a tetrapyrrole photocatalyst; and
a protein labeling agent, wherein the tetrapyrrole photocatalyst has electronic structure to activate the protein labeling agent to a reactive intermediate via energy transfer.

2. The composition of claim 1, wherein the tetrapyrrole photocatalyst absorbs electromagnetic radiation having wavelength greater than 600 nm.

3. The composition of claim 1, wherein the tetrapyrrole photocatalyst absorbs electromagnetic radiation having wavelength in the range of 600 nm to 1100 nm.

4. The composition of claim 1, wherein the reactive intermediate crosslinks with a protein.

5. The composition of claim 1, wherein the reactive intermediate inserts into a C—H bond of a protein.

6. The composition of claim 1, wherein the protein labeling agent comprises an azide, diazirine, phenol, thiatriazole, sulfilimine, sulfoximine, ylide, diazo, aniline, or mixtures thereof.

7. The composition of claim 1, wherein the reactive intermediate is an aminyl radical.

8. The composition of claim 1, wherein the tetrapyrrole photocatalyst comprises transition metal center.

9. The composition of claim 1, wherein tetrapyrrole photocatalyst comprises metalloid center.

10. The composition of claim 1, wherein the reactive intermediate has a diffusion radius less than 10 nm prior to quenching in an aqueous or aqueous-based environment.

11. The composition of claim 1, wherein the reactive intermediate has a diffusion radius of 1-5 nm prior to quenching in an aqueous or aqueous-based environment.

12. The composition of claim 1, wherein the energy transfer is single electron transfer.

13. The composition of claim 1, wherein the energy transfer is from a ground state of the teatrpyrrole photocatalyst.

14. The composition of claim 13, wherein the ground state is formed from a reduced excited state of the teatrpyrrole photocatalyst.

15. The composition of claim 1, wherein a biomolecular binding agent is coupled to the teatrpyrrole photocatalyst.

16. The composition of claim 1, wherein the biomolecular binding agent comprises a protein, polysaccharide, nucleic acid, or lipid.

17. The composition of claim 1, wherein the tetrapyrrole photocatalyst is soluble in water or aqueous solution.

18-23. (canceled)

24. A system for proximity-based labeling comprising:
a conjugate including a tetrapyrrole photocatalyst coupled to a biomolecular binding agent; and
a protein labeling agent, wherein the tetrapyrrole photocatalyst has electronic structure to activate the protein labeling agent to a reactive intermediate via energy transfer.

25. The system of claim 24, wherein the tetrapyrrole photocatalyst absorbs electromagnetic radiation having wavelength in the range of 600 nm to 1100 nm.

26. The system of claim 24, wherein the reactive intermediate crosslinks with a protein.

27. The system of claim 24, wherein the biomolecular binding agent comprises a ligand specific to a cell surface receptor, and the reactive intermediate crosslinks with the surface cell receptor.

28. The system of claim 24, wherein the reactive intermediate inserts into a C—H bond of a protein.

29. The system of claim 24 further comprising a reductant for the tetrapyrrole photocatalyst.

30. The system of claim 24, wherein the energy transfer is single electron transfer.

31. The system of claim 24, wherein the energy transfer is from a ground state of the photocatalyst.

32. The system of claim 24, wherein the biomolecular binding agent comprises a protein, polysaccharide, nucleic acid, or lipid.

33. The system of claim 24, wherein the protein labeling agent comprises an azide, diazirine, phenol, thiatriazole, sulfilimine, sulfoximine, ylide, diazo, aniline, or mixtures thereof.

34. The system of claim 24, wherein the tetrapyrrole photocatalyst comprises a metal center selected from the group consisting of a transition metal, metalloid, and alkaline earth metal.

35. The conjugate of claim 34, wherein the metal center is selected from the group consisting of tin and silicon.

36. A method of proximity-based labeling comprising:
providing a conjugate comprising a tetrapyrrole photocatalyst coupled to a biomolecular binding agent;
locating the conjugate in a targeted area with the biomolecular binding agent;
activating a protein labeling agent to a reactive intermediate with the tetrapyrrole photocatalyst; and
coupling the reactive intermediate to one or more proteins in the targeted area.

37. The method of claim 36, wherein the targeted area is in vivo.

38-45. (canceled)
